(19) United States
(12) Patent Application Publication
(10) Pub. No.: US 2010/0222580 A1
(43) Pub. Date: Sep. 2, 2010

(54) Process For The Preparation Of Onium Alkylsulfates Having A Low Halide Content

(75) Inventors: Nikolai Ignatyev, Duisburg (DE); Urs Welz-Biermann, Heppenheim (DE); Andriy Kucheryna, Wuppertal (DE); Helge Willner, Muelheim/Ruhr (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, Darmstadt (DE)

(21) Appl. No.: 11/721,610

(22) PCT Filed: Nov. 18, 2005
(86) PCT No.: PCT/EP2005/012399

§ 371 Date: Sep. 9, 2009

(30) Foreign Application Priority Data
Dec. 14, 2004 (DE) 10 2004 060 074.0

(51) Int. Cl. C07D 233/58; C07F 9/54; C07D 213/20; C07C 323/25; C07D 295/037; C07C 211/13; C07F 7/08; C07D 231/06; C07D 233/04; C07D 249/04; C07D 249/08; C07D 237/06; C07D 239/24; C07D 241/10; C07D 265/30; C07D 277/20; C07D 263/30

(52) U.S. Cl. 544/106; 548/335.1; 568/9; 546/347; 564/501; 548/579; 564/512; 556/465; 548/379.1; 548/347.1; 548/255; 548/262.2; 544/224; 544/242; 544/336; 548/202; 548/235

(57) ABSTRACT

The invention relates to a process for the preparation of onium alkylsulfates by reaction of an onium halide with a symmetrically substituted dialkyl sulfate, in which the alkyl group can have 1 to 14 C atoms, with an asymmetrically substituted dialkyl sulfate, in which one alkyl group can have 4 to 20 C atoms and the second alkyl group denotes methyl or ethyl, with an alkyl trialkylsilyl sulfate, with an alkyl acyl sulfate or with an alkyl sulfonyl sulfate, where the reaction with a dialkyl sulfate is carried out at room temperature.

The invention relates to a process for the preparation of onium alkylsulfates by reaction of an onium halide with a symmetrically substituted dialkyl sulfate, in which the alkyl group can have 1 to 14 C atoms, with an asymmetrically substituted dialkyl sulfate, in which one alkyl group can have 4 to 20 C atoms and the second alkyl group denotes methyl or ethyl, with an alkyl trialkylsilyl sulfate, with an alkyl acyl sulfate or with an alkyl sulfonyl sulfate, where the reaction with a dialkyl sulfate is carried out at room temperature.

A large number of onium salts, in particular alkyl sulfates, are ionic liquids. Owing to their properties, ionic liquids represent an effective alternative to traditional volatile organic solvents for organic synthesis in modern research. The use of ionic liquids as novel reaction medium could furthermore be a practical solution both for solvent emission and also for problems in the reprocessing of catalysts.

Ionic liquids or liquid salts are ionic species which consist of an organic cation and a generally inorganic anion. They do not contain any neutral molecules and usually have melting points below 373 K. However, the melting point may also be higher without restricting the usability of the salts in all areas of application. Examples of organic cations are, inter alia, tetraalkylammonium, tetraalkylphosphonium, N-alkylpyridinium, 1,3-dialkylimidazolium or trialkylsulfonium. Amongst a multiplicity of suitable anions, mention may be made, for example, of BF₄⁻, PF₆⁻, SbF₆⁻, NO₃⁻, CF₃SO₃⁻, (CF₃SO₂)₂N⁻, arylSO₃⁻, CF₃CO₂⁻, CH₃CO₂⁻ or Al₂Cl₇⁻.

A general method for the preparation of onium alkylsulfates is alkylation of the organic base, i.e., for example, the amine, phosphine, guanidine or heterocyclic base, using dialkyl sulfates, also disclosed by the publication by John D. Holbrey et al, Green Chemistry (2002), 4 (5), 407-413. However, a disadvantage of this method is that one substituent of the onium alkylsulfate formed always corresponds to the corresponding alkyl group of the dialkyl sulfate. For the preparation of onium alkylsulfates whose alkyl group in the anion is different from the substituents of the cation, referred to below as asymmetrically substituted onium alkylsulfates, it would be necessary to employ asymmetrically substituted dialkyl sulfates, for example ethyl methyl sulfate, which result in mixed-alkylated onium alkylsulfates. On the one hand the organic base would be ethylated giving a methyl sulfate, on the other hand the organic base would be methylated giving an ethyl sulfate.

Asymmetric onium alkylsulfates, as defined above, for example 1-butyl-3-methylimidazolium octylsulfate, can be synthesised by the method of P. Wasserscheid et al, Green Chemistry (2002), 4 (4), 400-404, also by reaction of the onium halide, for example 1-butyl-3-methylimidazolium chloride, with a corresponding alkali metal sulfate, for example sodium octylsulfate, but the alkali metal halide formed, for example sodium chloride, has to be removed by an additional purification method. Contamination by halide ions, for example chloride ions, of greater than 1000 ppm (0.1%), reduces the usability of the ionic liquid, in particular in the application for electrochemical processes. The technology is therefore of crucial importance in processes for the preparation of onium salts, in particular ionic liquids, in order that they can be synthesised with a low level of impurities by the reaction per se or by the reaction procedure, and thus further expensive additional process steps during the synthesis are superfluous.

The object of the present invention was accordingly to provide an alternative process for the preparation of onium alkylsulfates having a low halide content which results in alkyl sulfates, preferably asymmetrically substituted onium alkylsulfates, of high purity in good yield and is also suitable for large-scale industrial production.

A process of this type is of course then also suitable for the preparation of symmetrically substituted onium alkylsulfates. The method according to the invention can also be used for the purification of halide-containing onium alkylsulfates.

The object is achieved by the process according to the invention since the symmetrically substituted dialkyl sulfate, in which the alkyl groups can have 1 to 14 C atoms, the asymmetrically substituted dialkyl sulfate, in which one alkyl group can have 4 to 20 C atoms and the second alkyl group denotes methyl or ethyl, the alkyl trialkylsilyl sulfate, the alkyl acyl sulfate or alkyl sulfonyl sulfate alkylates the anion of the onium halide employed and not the organic cation. The alkyl, acyl, trialkylsilyl or sulfonyl halides formed as by-product are generally gases or readily volatile compounds which can be removed from the reaction mixture without major process-engineering measures.

The invention therefore relates to a process for the preparation of onium alkylsulfates, in particular asymmetrically substituted onium alkylsulfates, by reaction of an onium halide with a symmetrically substituted dialkyl sulfate, in which the alkyl group can have 1 to 14 C atoms, with an asymmetrically substituted dialkyl sulfate, in which one alkyl group can have 4 to 20 C atoms and the second alkyl group denotes methyl or ethyl, with an alkyl trialkylsilyl sulfate, with an alkyl acyl sulfate or with an alkyl sulfonyl sulfate, where the reaction with a dialkyl sulfate is carried out at room temperature.

On use of a C₄-C₂₀-alkyl methyl sulfate or C₄-C₂₀-alkyl ethyl sulfate, the formation of mixtures is avoided since the methyl group or ethyl group is more reactive and will methylate or ethylate the halide, and the alkyl sulfate having 4 to 20 C atoms mostly forms the anion of the onium alkylsulfate.

It is of course possible to use both symmetrically substituted dialkyl sulfates having 1 to 20 C atoms or to use asymmetrically substituted dialkyl sulfates having 1 to 20 C atoms or even more highly alkylated starting materials in the process according to the invention. The advantage is always that the onium alkylsulfates formed have been prepared with a reduced halide content.

U.S. Pat. No. 2,585,979 discloses the preparation of quaternary sulfates of pyrimidylaminoquinoline derivatives

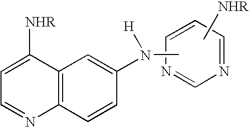

by reaction of the corresponding pyrimidylaminoquinoline halide with dimethyl or diethyl sulfate. In contrast to the process according to the invention, however, the reaction is carried out at temperatures of 90° to 150° C. in the presence of a solvent, for example nitrobenzene. Surprisingly, however, the reaction according to the invention succeeds at room temperature, i.e. at temperatures between 10° and 30° C., without the use of a solvent, is approximately quantitative yield.

The same technical teaching of U.S. Pat. No. 2,585,979 is also provided in Vompe et al, J. Org. Chem. USSR (Engl. transl), 17, 1981, 1551-1554, where it is disclosed that the reaction of 2-methyl-3-ethylnaphtho[2,1-d]thiazolium iodide with dimethyl sulfate at 80°-130° C. gives a mixture of methyl sulfate and the bisulfate (hydrogensulfate), whereas at temperatures of 130° C. the bisulfate HSO₄⁻ is obtained. Here too, high temperatures are required.

On use of, in particular, symmetrically substituted dialkyl sulfates as reagent, the process according to the invention should therefore be regarded as a selection invention from the processes of the prior art. There is no indication of the use of the reagents alkyl trialkylsilyl sulfate, alkyl acyl sulfate or alkyl sulfonyl sulfate.

Suitable onium halides are ammonium halides, phosphonium halides, thiouronium halides, guanidinium halides or halides with a heterocyclic cation, where the halides can be selected from the group chlorides or bromides. Preference is given in the process according to the invention to the use of phosphonium, thiouronium or guanidinium halides or halides with a heterocyclic cation. Besides the chlorides and bromides, the thiouronium iodides are particularly suitable.

The onium halides are generally commercially available or can be prepared by synthetic methods as known from the literature, for example in the standard works, such as Houben-Weyl, Methoden der organischen Chemie [Methods of Organic Chemistry], Georg-Thieme-Verlag, Stuttgart or Richard C. Larock, Comprehensive Organic Transformations, 2nd Edition, Wiley-VCH, New York, 1999. Use can also be made here of variants known per se which are not mentioned here in greater detail.

Phosphonium halides can be described, for example, by the formula (1)

[PR₄]⁺Hal⁻ (1),

where
Hal denotes Cl or Br and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
where one or more R may be partially or fully substituted by halogens, in particular —F and/or —Cl, or partially by —NO₂, but where all four or three R must not be fully substituted by halogens.

Accordingly, compounds of the formula (1) in which all four or three substituents R are fully substituted by halogens, for example tris(trifluoromethyl)methylphosphonium chloride, tetra(trifluoromethyl)phosphonium chloride or tetra(nonafluorobutyl)phosphonium chloride, are excluded.

Guanidinium halides can be described, for example, by the formula (2)

[C(NR¹R²)(NR³R⁴)(NR⁵R⁶)]⁺Hal⁻ (2),

where
Hal denotes Cl or Br and
R¹ to R⁶ each, independently of one another, denotes
hydrogen,
straight-chain or branched alkyl having 1 to 20 C atoms,
straight-chain or branched alkenyl having 2-20 C atoms and one or more double bonds,
where one or more of the substituents R¹ to R⁶ may be partially or fully substituted by halogens, in particular —F and/or —Cl, or partially by —NO₂, but where all substituents on one N atom must not be fully substituted by halogens and
where the substituents R¹ to R⁶ may be connected to one another in pairs by a single or double bond.

Thiouronium halides can be described, for example, by the formula (3)

[(R¹R²N)—C(═SR⁷)(NR³R⁴)]⁺Hal⁻ (3),

where
Hal denotes Cl, Br or I and
R¹ to R⁷ each, independently of one another, denotes
hydrogen, where hydrogen is excluded for R⁷,
straight-chain or branched alkyl having 1 to 20 C atoms,
straight-chain or branched alkenyl having 2-20 C atoms and one or more double bonds,
where one or more of the substituents R¹ to R⁷ may be partially or fully substituted by halogens, in particular —F and/or —Cl, or partially by —NO₂, but where all substituents on one N atom must not be fully substituted by halogens and
where the substituents R¹ to R⁷ may be connected to one another in pairs by a single or double bond.

Halides with a heterocyclic cation can be described, for example, by the formula (4)

[HetN]⁺Hal⁻ (4),

where
Hal denotes Cl or Br and
HetN⁺ denotes a heterocyclic cation selected from the group

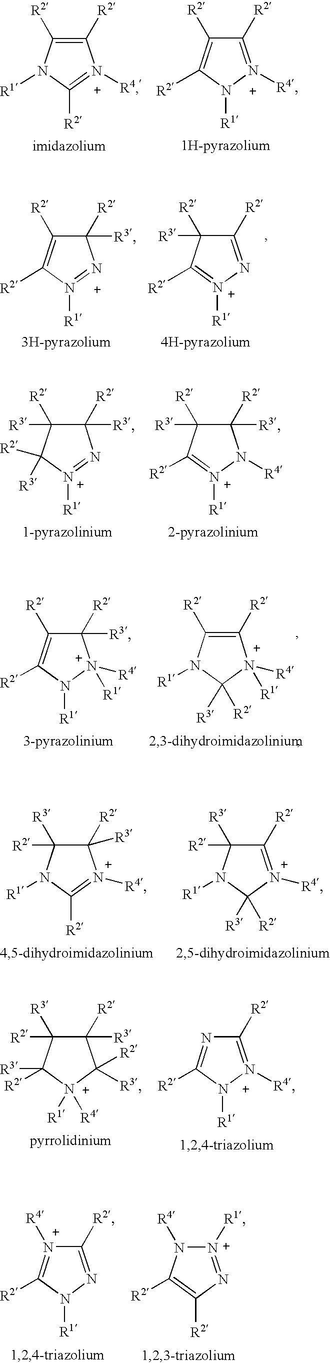

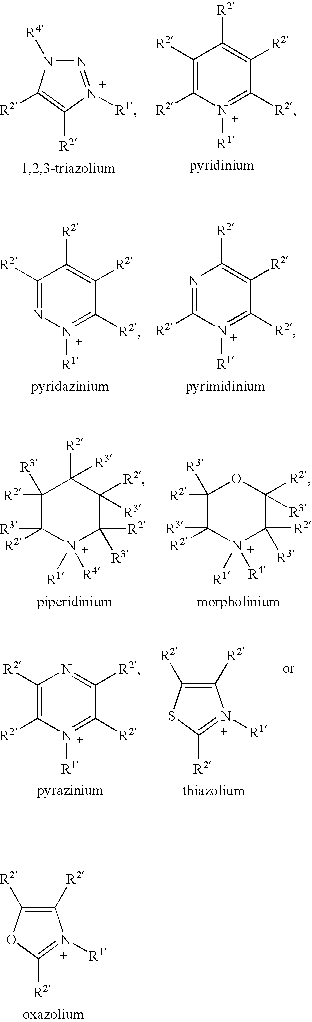

where the substituents
R^{1′} to R^{4′} each, independently of one another, denote
hydrogen or CN,
straight-chain or branched alkyl having 1-20 C atoms,
straight-chain or branched alkenyl having 2-20 C atoms and one or more double bonds or
aryl-C₁-C₆-alkyl,
where one or more substituents R^{1′} to R^{4′} may be partially or fully substituted by halogens, in particular —F and/or —Cl, or partially by —NO₂ or CN, but where R^{1′} and R^{4′} must not simultaneously be fully substituted by halogens.

The C₁-C₁₄-alkyl group is, for example, methyl, ethyl, isopropyl, propyl, butyl, sec-butyl or tert-butyl, furthermore also pentyl, 1-, 2- or 3-methylbutyl, 1,1-, 1,2- or 2,2-dimethylpropyl, 1-ethylpropyl, hexyl, heptyl, octyl, nonyl, decyl, undecyl, dodecyl, tridecyl or tetradecyl, or optionally perfluorinated alkyl groups, for example difluoromethyl, trifluoromethyl, pentafluoroethyl, heptafluoropropyl or nonafluorobutyl.

A straight-chain or branched alkenyl having 2 to 20 C atoms, in which, in addition, a plurality of double bonds may be present, is, for example, vinyl, allyl, 2- or 3-butenyl, isobutenyl, sec-butenyl, furthermore 4-pentenyl, isopentenyl, hexenyl, heptenyl, octenyl, —C₉H₁₇, —C₁₀H₁₉ to —C₂₀H₃₉; preferably vinyl, allyl, 2- or 3-butenyl, isobutenyl, sec-butenyl, furthermore preferably 4-pentenyl, isopentenyl or hexenyl.

Aryl-C₁-C₆-alkyl denotes, for example, benzyl, phenylethyl, phenylpropyl, phenylbutyl, phenylpentyl or phenylhexyl, in which both the phenyl ring and also the alkylene chain may, as described above, be partially or fully substituted by halogens, in particular —F and/or —Cl, or partially by —NO₂, particularly preferably benzyl or phenylpropyl. However, the phenyl ring or also the alkylene chain may likewise be substituted by further functional groups, for example by CN, SO₂R′, SO₂OR′ or COOR′. R′ here has a meaning defined above.

In accordance with the invention, suitable substituents R and R¹ to R⁷ of the compounds of the formulae (1) to (3) are, besides hydrogen, preferably in each case, independently of one another, where hydrogen is excluded for R⁷: C₁- to C₂₀-, in particular C₁- to C₁₄-alkyl groups.

However, the substituents R and R¹ to R⁷ may likewise be substituted by further functional groups, for example by CN, SO₂R′, SO₂OR′ or COOR′. R′ denotes non-, partially or perfluorinated C₁- to C₆-alkyl, C₃- to C₇-cycloalkyl, unsubstituted or substituted phenyl.

The alkyl groups as substituents R and R¹ to R⁶ and R^{1′} and R^{4′} of the heterocyclic cations of the formula (4) are preferably different from the alkyl group of the anion in the onium alkylsulfate.

However, the onium alkylsulfate prepared in accordance with the invention may also have alkyl groups in the cation which are identical with the alkyl group in the anion, but have not been introduced in accordance with the invention by alkylation. The focus is then on the simple reaction procedure and the particularly low halide content in the end product.

The substituent R in formula (1) is in particular, in each case independently of one another, preferably methyl, ethyl, isopropyl, propyl, butyl, sec-butyl, tert-butyl, pentyl, hexyl, octyl, decyl or tetradecyl.

Up to four substituents of the guanidinium cation [C(NR¹R²)(NR³R⁴)—(NR⁵R⁶)]⁺ may also be connected in pairs in such a way that mono-, bi- or polycyclic cations are formed.

Without restricting generality, examples of such guanidinium cations are:

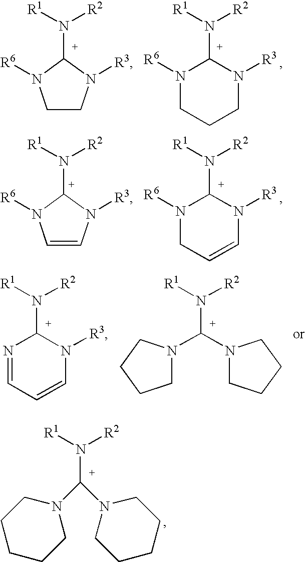

where the substituents R¹ to R³ and R⁶ may have an above-mentioned or particularly preferred meaning.

The carbocycles or heterocycles of the above-mentioned guanidinium cations may optionally also be substituted by C₁- to C₆-alkyl, C₁- to C₆-alkenyl, NO₂, F, Cl, Br or I.

Up to four substituents of the thiouronium cation [(R¹R²N)—C(═SR⁷)—(NR³R⁴)]⁺ may also be connected in pairs in such a way that mono-, bi- or polycyclic cations are formed.

Without restricting generality, examples of such cations are indicated below:

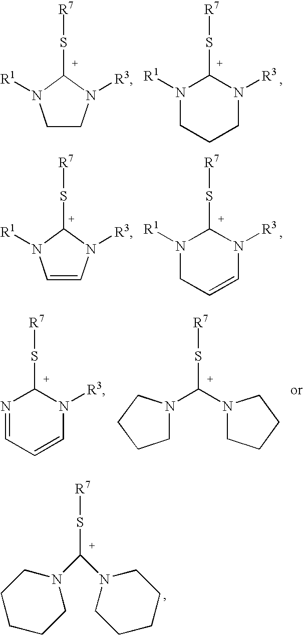

where the substituents R¹, R³ and R⁷ may have an above-mentioned or particularly preferred meaning.

The carbocycles or heterocycles of the above-mentioned thiouronium cations may optionally also be substituted by C₁- to C₆-alkyl, C₁- to C₆-alkenyl, NO₂, F, Cl, Br, I, C₁-C₆-alkoxy, SCF₃, SO₂CF₃, SO₂CH₃, COOR″, SO₂NR″₂, SO₂X′ or SO₃R″, where X′ denotes F, Cl or Br and R″ denotes a non-, partially or perfluorinated C₁- to C₆-alkyl or C₃- to C₇-cycloalkyl as defined for R′, or by substituted or unsubstituted phenyl.

The substituents R¹ to R⁷ are each, independently of one another, preferably a straight-chain or branched alkyl group having 1 to 10 C atoms. The substituents R¹ and R², R³ and R⁴ and R⁵ and R⁶ in compounds of the formulae (2) or (3) may be identical or different here.

R¹ to R⁷ are particularly preferably each, independently of one another, methyl, ethyl, n-propyl, isopropyl, n-butyl, tert-butyl or sec-butyl, very particularly preferably methyl, ethyl, n-propyl, isopropyl or n-butyl.

In accordance with the invention, suitable substituents R^{1′} to R^{4′} of compounds of the formula (4), besides hydrogen, are preferably: C₁- to C₂₀-, in particular C₁- to C₁₂-alkyl groups or aryl-C₁-C₆-alkyl.

However, the substituents R^{1′} to R^{4′} may likewise be substituted by further functional groups, for example by CN, SO₂R′, SO₂OR′ or COOR′. R′ denotes non-, partially or perfluorinated C₁- to C₆-alkyl, C₃- to C₇-cycloalkyl, unsubstituted or substituted phenyl.

The substituents R^{1′} and R^{4′} are each, independently of one another, particularly preferably methyl, ethyl, isopropyl, propyl, butyl, sec-butyl, tert-butyl, pentyl, hexyl, octyl, decyl, undecyl, dodecyl or benzyl. They are very particularly preferably methyl, ethyl, n-butyl, hexyl or benzyl. In pyrrolidinium or piperidinium compounds, the two substituents R^{1′} and R^{4′} are preferably different.

The substituent R^{2′} or R^{3′} is in each case, independently of one another, in particular hydrogen, methyl, ethyl, isopropyl, propyl, butyl, sec-butyl or tert-butyl. R^{2′} is particularly preferably hydrogen, methyl or ethyl. R^{2′} and R^{3′} are very particularly preferably hydrogen.

HetN⁺ of the formula (4) is preferably

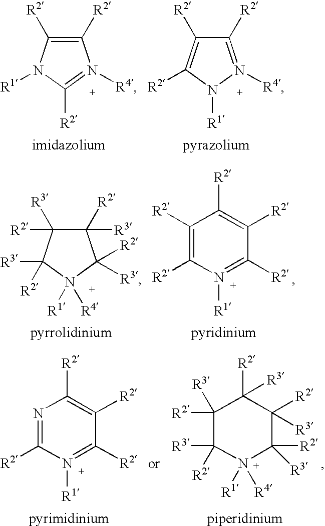

where the substituents R^{1′} to R^{4′} each, independently of one another, have a meaning described above.

HetN⁺ is particularly preferably imidazolium, pyrrolidinium or pyridinium, as defined above, where the substituents R^{1′} to R^{4′} each, independently of one another, have a meaning described above.

The symmetrically substituted dialkyl sulfate employed is preferably a dialkyl sulfate having a straight-chain or branched alkyl group having 1-14 C atoms, preferably having 1-8 C atoms. Examples of symmetrically substituted dialkyl sulfates are dimethyl sulfate, diethyl sulfate, di(n-propyl) sulfate, di(isopropyl) sulfate, di(n-butyl) sulfate, di(sec-butyl) sulfate and di(n-pentyl) sulfate, di(n-hexyl) sulfate, di(n-heptyl) sulfate and di(n-octyl) sulfate.

The symmetrical dialkyl sulfates employed are generally commercially available or can be prepared by synthetic methods as known from the literature, for example in the standard works, such as Houben-Weyl, Methoden der organischen Chemie [Methods of Organic Chemistry], Georg-Thieme-Verlag, Stuttgart, or Richard C. Larock, Comprehensive Organic Transformations, 2nd Edition, Wiley-VCH, New York, 1999. Use can also be made here of variants known per se which are not mentioned here in greater detail.

The asymmetrically substituted dialkyl sulfate employed is preferably a dialkyl sulfate having a straight-chain or branched alkyl group having 4 to 20 C atoms and a methyl or ethyl group as the second alkyl group, preferably having an alkyl group having 4-8 C atoms. Examples of asymmetrically substituted dialkyl sulfates are methyl butyl sulfate, ethyl butyl sulfate, methyl pentyl sulfate, ethyl pentyl sulfate, methyl hexyl sulfate, ethyl hexyl sulfate, methyl heptyl sulfate, ethyl heptyl sulfate, methyl octyl sulfate and ethyl octyl sulfate.

The asymmetric dialkyl sulfates employed can be prepared by synthetic methods as known from the literature, for example in the standard works, such as Houben-Weyl, Methoden der organischen Chemie [Methods of Organic Chemistry], Georg-Thieme-Verlag, Stuttgart, or Richard C. Larock, Comprehensive Organic Transformations, 2nd Edition, Wiley-VCH, New York, 1999. Use can also be made here of variants known per se which are not mentioned here in greater detail.

Alkyl trialkylsilyl sulfates conform to the formula alkyl-O—SO₂—OSi(alkyl′)₃, in which the alkyl group can have 1 to 20 C atoms and the alkyl′ group can have 1 to 4 C atoms. The alkyl′ groups are preferably identical. Alkyl′ is preferably methyl.

The alkyl trialkylsilyl sulfates employed can be prepared by synthetic methods as known from the literature, for example in the standard works, such as Houben-Weyl, Methoden der organischen Chemie [Methods of Organic Chemistry], Georg-Thieme-Verlag, Stuttgart or Richard C. Larock, Comprehensive Organic Transformations, 2nd Edition, Wiley-VCH, New York, 1999. Use can also be made here of variants known per se which are not mentioned here in greater detail.

Alkyl acyl sulfates conform to the formula alkyl-O—SO₂—O—C(O)R^{F}, in which the alkyl group can have 1 to 20 C atoms and the R^{F} group denotes a perfluoroalkyl group having 1 to 4 C atoms. R^{F} is preferably trifluoromethyl.

The alkyl acyl sulfates employed can be prepared by synthetic methods as known from the literature, for example in the standard works, such as Houben-Weyl, Methoden der organischen Chemie [Methods of Organic Chemistry], Georg-Thieme-Verlag, Stuttgart or Richard C. Larock, Comprehensive Organic Transformations, 2nd Edition, Wiley-VCH, New York, 1999. Use can also be made here of variants known per se which are not mentioned here in greater detail.

Alkyl sulfonyl sulfates conform to the formula alkyl-O—SO₂—O—SO₂R^{F′}, in which the alkyl group can have 1 to 20 C atoms and the R^{F′} group denotes a perfluoroalkyl group having 1 to 4 C atoms, Cl or F. R^{F′} is preferably F or trifluoromethyl.

The alkyl sulfonyl sulfates employed can be prepared by synthetic methods as known from the literature, for example in the standard works, such as Houben-Weyl, Methoden der organischen Chemie [Methods of Organic Chemistry], Georg-Thieme-Verlag, Stuttgart or Richard C. Larock, Comprehensive Organic Transformations, 2nd Edition, Wiley-VCH, New York, 1999). Use can also be made here of variants known per se which are not mentioned here in greater detail.

A general scheme summarises the process according to the invention:

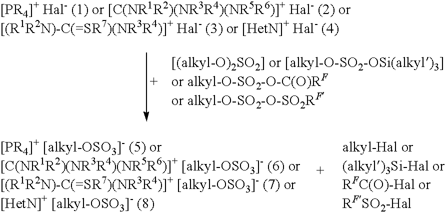

The substituents R, R¹ to R⁷ and HetN⁺ of the compounds of the formulae (1) to (8) correspond to the meanings as described above.

The reaction with dialkyl sulfates is carried out in accordance with the invention at room temperature, i.e. generally at temperatures between 10° and 30° C. The reaction with alkyl trialkylsilyl sulfates, alkyl acyl sulfates or alkyl sulfonyl sulfates can be carried out at temperatures between 0° and 200° C., preferably at 10° to 100°, particularly preferably at 10° to 50° C., very particularly preferably at room temperature, where the temperatures from 50° C. corresponds to the temperature of the heating source, for example the oil bath. No solvent is required. However, it is also possible to employ solvents, for example dimethoxyethane, acetonitrile, acetone, tetrahydrofuran, dimethylformamide, dimethyl sulfoxide, dioxane, propionitrile or mixtures with one another.

The reaction is carried out with an excess or equimolar amount of dialkyl sulfate, alkyl trialkylsilyl sulfate, alkyl acyl sulfate or alkyl sulfonyl sulfate.

The method described is also suitable for the purification of the onium salts. This means that a corresponding ester according to the invention or a silyl ester of the acid of the anion, for example with dimethyl sulfate, is added to the ionic liquid contaminated by halide ions, for example 1-butyl-3-methylimidazolium methylsulfate contaminated with 1-butyl-3-methylimidazolium chloride. The contamination reacts away, and the halide-reduced ionic liquid is obtained.

The invention likewise relates to a one-pot process for the preparation of onium alkylsulfates, in particular alkylsulfates in which the alkyl group has 4 to 20 C atoms, particularly preferably 4 to 14 C atoms, characterised in that an onium halide is reacted with a symmetrically substituted dialkyl sulfate, in which the alkyl group can have 1 to 3 C atoms, and with an alcohol having 4 to 20 C atoms.

The by-products alkyl halide having 1 to 3 C atoms and the alcohol having 1 to 3 C atoms can easily be removed.

The reaction is carried out at temperatures between 0° and 200° C., preferably at 10° to 100°, particularly preferably at 10° to 60° C., and with use of vacuum, where the temperatures from 60° C. corresponds to the temperature of the heating source, for example the oil bath.

The one-pot reaction is particularly preferably carried out with the symmetrical dimethyl sulfate and the alcohols hexanol, heptanol or octanol, very particularly preferably octanol.

The invention also relates to the compounds trialkylsilyl octyl sulfate, in which the alkyl group of the trialkylsilyl group can have 1 to 4 C atoms. Preferred trialkylsilyl octyl sulfates are compounds whose alkyl group in the trialkylsilyl group is identical. Particular preference is given to trimethylsilyl octyl sulfate or triethylsilyl octyl sulfate, very particularly preferably trimethylsilyl octyl sulfate.

These compounds are highly suitable for use in the process according to the invention, i.e. for the introduction of octyl sulfate anions into ionic liquids.

Even without further comments, it is assumed that a person skilled in the art will be able to utilise the above description in the broadest scope. The preferred embodiments and examples should therefore merely be regarded as descriptive disclosure which is absolutely not limiting in any way.

It goes without saying for the person skilled in the art that substituents in the compounds mentioned above and below, such as, for example, H, N, O, Cl or F, can be replaced by the corresponding isotopes.

The NMR spectra were measured on solutions in deuterated solvents at 20° C. on a Bruker ARX 400 spectrometer with a 5 mm ¹H/BB broadband head with deuterium lock, unless indicated in the examples. The measurement frequencies of the various nuclei are: ¹H, 400.13 MHz and ¹⁹F: 376.50 MHz. The referencing method is indicated separately for each spectrum or each data set.

### EXAMPLE 1

### Synthesis of 1-butyl-3-methylimidazolium ethylsulfate

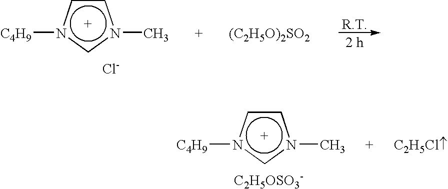

A mixture of 3.62 g (20.7 mmol) of 1-butyl-3-methylimidazolium chloride and 3.19 g (20.7 mmol) of diethyl sulfate is stirred at room temperature for two hours. NMR measurements show the completeness of the reaction. The residue is dried for 30 minutes in vacuo at 13.3 Pa and 60° C. (oil-bath temperature), giving 5.47 g of 1-butyl-3-methylimidazolium ethylsulfate in approximately quantitative yield.

¹H NMR (reference: TMS; CD₃CN), ppm: 0.92 t (CH₃); 1.18 t (CH₃); 1.31 m (CH₂); 1.81 m (CH₂); 3.86 s (CH₃); 3.88 q (CH₂); 4.16 t (CH₂); 7.42 d, d (CH); 7.45 d, d (CH); 8.88 br. s. (CH); ³J_{H,H}=7.4 Hz; ³J_{H,H}=7.2; ³J_{H,H}=7.1 Hz; J_{H,H}=1.7 Hz.

### EXAMPLE 2

### Synthesis of 1-hexyl-3-methylimidazolium methylsulfate

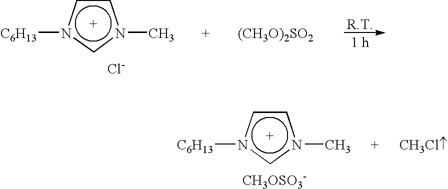

Analogously to Example 1, 1.51 g (7.45 mmol) of 1-hexyl-3-methylimidazolium chloride and 1.02 g (8.09 mmol) of dimethyl sulfate are stirred for one hour and dried in vacuo at 13.3 Pa and 120° C. (oil-bath temperature), giving 2.06 g of 1-hexyl-3-methylimidazolium methylsulfate in approximately quantitative yield.

¹H NMR (reference: TMS; CD₃CN), ppm: 0.86 m (CH₃); 1.28 m (3CH₂); 1.81 m (CH₂); 3.50 s (OCH₃); 3.84 s (CH₃); 4.13 t (CH₂); 7.39 d, d (CH); 7.42 d, d (CH); 8.81 br. s. (CH); ³J_{H,H}7.1 Hz; J_{H,H}=1.5 Hz.

### EXAMPLE 3

### Synthesis of 1-butyl-3-methylimidazolium methylsulfate

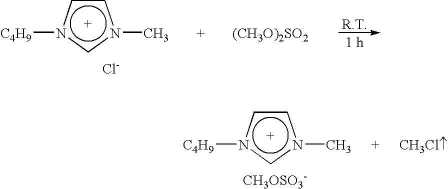

Analogously to Example 1, 1.36 g (7.79 mmol) of 1-butyl-3-methylimidazolium chloride and 0.99 g (7.95 mmol) of dimethyl sulfate are stirred for one hour and dried in vacuo at 13.3 Pa and 120° C. (oil-bath temperature), giving 1.95 g of 1-butyl-3-methylimidazolium methylsulfate in approximately quantitative yield.

¹H NMR (reference: TMS; CD₃CN), ppm: 0.90 t (CH₃); 1.29 m (CH₂); 1.79 m (CH₂); 3.50 s (OCH₃); 3.84 s (CH₃); 4.15 t (CH₂); 7.40 d, d (CH); 7.43 d, d (CH); 8.91 br. s. (CH); ³J_{H,H}=7.4 Hz; ³J_{H,H}=7.1 Hz; J_{H,H}=1.7 Hz.

### EXAMPLE 4

Analogously to Example 3,

1,3-dimethylimidazolium chloride is reacted with dimethyl sulfate to give
1,3-dimethylimidazolium methylsulfate;
   1,3-dibutylimidazolium chloride is reacted with dimethyl sulfate to give
1,3-dibutylimidazolium methylsulfate;
   1-ethyl-3-methylimidazolium chloride is reacted with dimethyl sulfate to give
1-ethyl-3-methylimidazolium methylsulfate;
   1-ethyl-3-methylimidazolium chloride is reacted with diethyl sulfate to give
1-ethyl-3-methylimidazolium ethylsulfate;
   1-ethyl-3-methylimidazolium chloride is reacted with dibutyl sulfate to give
1-ethyl-3-methylimidazolium butylsulfate;
   1-ethyl-3-methylimidazolium chloride is reacted with dihexyl sulfate to give
1-ethyl-3-methylimidazolium hexylsulfate;
   1-ethyl-3-methylimidazolium chloride is reacted with dioctyl sulfate to give
1-ethyl-3-methylimidazolium octylsulfate;
   1-butyl-3-methylimidazolium chloride is reacted with dioctyl sulfate to give
1-butyl-3-methylimidazolium octylsulfate;
   3-methyl-1-octylimidazolium chloride is reacted with dioctyl sulfate to give
3-methyl-1-octylimidazolium octylsulfate;
   3-methyl-1-octylimidazolium chloride is reacted with dimethyl sulfate to give
3-methyl-1-octylimidazolium methylsulfate;
   1-benzyl-3-methylimidazolium chloride is reacted with dimethyl sulfate to give
1-benzyl-3-methylimidazolium methylsulfate;
   1-ethyl-2,3-dimethylimidazolium chloride is reacted with dimethyl sulfate to give
1-ethyl-2,3-dimethylimidazolium methylsulfate;
   1-butyl-2,3-dimethylimidazolium chloride is reacted with dimethyl sulfate to give
1-butyl-2,3-dimethylimidazolium methylsulfate;
   1-butyl-2,3-dimethylimidazolium chloride is reacted with dioctyl sulfate to give
1-butyl-2,3-dimethylimidazolium octylsulfate.

### EXAMPLE 5

### Synthesis of trihexyltetradecylphosphonium methylsulfate

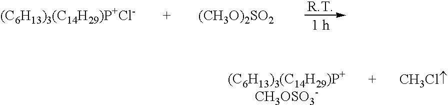

A mixture of 1.72 g (3.31 mmol) of trihexyltetradecylphosphonium chloride and 0.51 g (4.04 mmol) of dimethyl sulfate is stirred at room temperature for one hour. NMR measurements indicate the completeness of the reaction.

The residue is dried for 30 minutes in a vacuum of 13.3 Pa and at 120° C. (oil-bath temperature), giving 1.96 g of trihexyltetradecylphosphonium methylsulfate in approximately quantitative yield.

¹H NMR (reference: TMS; CD₃CN), ppm: 0.85-0.93 m (4CH₃), 1.24-1.37 m (16CH₂), 1.37-1.59 m (8CH₂), 2.02-2.12 m (4CH₂); 3.54 s (OCH₃).

³¹P {¹H} NMR (reference: 85% H₃PO₄—external; CD₃CN), ppm: 33.3.

### EXAMPLE 6

Analogously to Example 5, tributylmethylphosphonium chloride is reacted with diethyl sulfate to give tributylmethylphosphonium ethylsulfate.

### EXAMPLE 7

### Synthesis of 1-ethylpyridinium methylsulfate

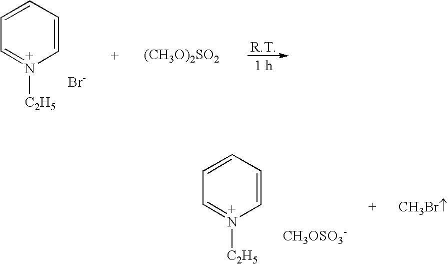

A mixture of 1.96 g (10.4 mmol) of 1-ethylpyridinium bromide and 1.31 g (10.4 mmol) of dimethyl sulfate is stirred at room temperature for one hour. NMR measurements indicate the completeness of the reaction. The residue is dried for 30 minutes in a vacuum of 13.3 Pa and at 120° C. (oil-bath temperature), giving 2.28 g of 1-ethylpyridinium methylsulfate in approximately quantitative yield.

¹H NMR (reference: TMS; CD₃CN), ppm: 1.57 t (CH₃); 3.50 s (OCH₃); 4.63 q (CH₂); 8.04 m (2CH); 8.50 t (CH); 8.92 d (2CH); ³J_{H,H}=7.1 Hz; ³J_{H,H}=7.9 Hz; ³J_{H,H}=6.1 Hz.

### EXAMPLE 8

### Synthesis of 1-butylpyridinium methylsulfate

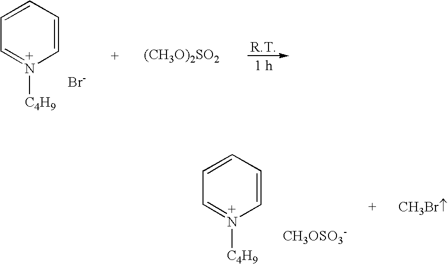

A mixture of 1.22 g (5.65 mmol) of 1-butylpyridinium bromide and 0.95 g (7.53 mmol) of dimethyl sulfate is stirred at room temperature for one hour. NMR measurements indicate the completeness of the reaction. The residue is dried for 30 minutes in a vacuum of 13.3 Pa and at 120° C. (oil-bath temperature), giving 1.39 g of 1-butylpyridinium methylsulfate in approximately quantitative yield.

¹H NMR (reference: TMS; CD₃CN), ppm: 0.92 t (CH₃); 1.34 m (CH₂); 1.93 m (CH₂); 3.50 s (OCH₃); 4.58 t (CH₂); 8.04 m (2CH); 8.50 t (CH); 8.88 d (2CH); ³J_{H,H}7.3 Hz; ³J_{H,H}=7.6 Hz; ³J_{H,H}=7.9 Hz; ³J_{H,H}6.6 Hz.

Analogously thereto,

1-butyl-3-methylpyridinium bromide is reacted with dimethyl sulfate to give
1-butyl-3-methylpyridinium methylsulfate;
   1-butyl-3-ethylpyridinium bromide is reacted with dimethyl sulfate to give
1-butyl-3-ethylpyridinium bromide methylsulfate;
   1-butyl-4-methylpyridinium chloride is reacted with dimethyl sulfate to give
1-butyl-4-methylpyridinium methylsulfate or
   1-butyl-4-ethylpyridinium chloride is reacted with dimethyl sulfate to give
1-butyl-4-ethylpyridinium methylsulfate.

### EXAMPLE 9

### Synthesis of 1-ethyl-1-methylpyrrolidinium ethylsulfate

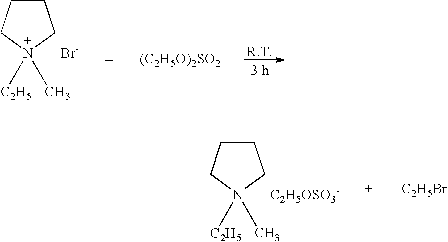

A mixture of 2.35 g (12.11 mmol) of 1-ethyl-1-methylpyrrolidinium bromide and 1.87 g (12.13 mmol) of diethyl sulfate is stirred at room temperature for three hours. NMR measurements indicate the completeness of the reaction. The residue is dried for one hour in a vacuum of 13.3 Pa and at room temperature, giving 2.89 g of 1-ethyl-1-methylpyrrolidinium ethylsulfate in approximately quantitative yield.

M.p.: 35-36° C.

¹H NMR (reference: TMS; CD₃CN), ppm: 1.16 t (CH₃); 1.31 t, m (CH₃); 2.14 m (2CH₂); 2.95 s (CH₃); 3.37 q (CH₂); 3.44 m (2CH₂); 3.84 q (CH₂); ³J_{H,H}=7.1 Hz.

Analogously thereto,

1-butyl-1-methylpyrrolidinium bromide is reacted with diethyl sulfate to give
1-butyl-1-methylpyrrolidinium ethylsulfate.

### EXAMPLE 10

### Synthesis of N,N,N′,N′-tetramethyl-N″-ethylguanidinium methylsulfate

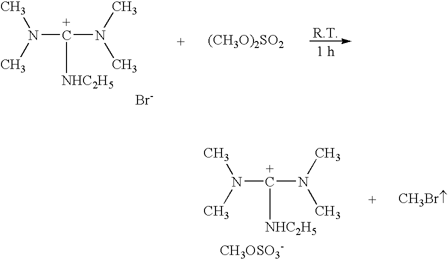

A mixture of 2.59 g (11.56 mmol) of N,N,N′,N′-tetramethyl-N″-ethyl-guanidinium bromide and 1.46 g (11.58 mmol) of dimethyl sulfate is stirred at room temperature for one hour. NMR measurements indicate the completeness of the reaction. The residue is dried for two hours in a vacuum of 13.3 Pa and at room temperature, giving 2.95 g of N,N,N′,N′-tetramethyl-N″-ethylguanidinium methylsulfate as a viscous liquid in approximately quantitative yield.

¹H NMR (reference: TMS; CD₃CN), ppm: 1.11 t (CH₃); 2.86 br.s; 2.88 br.s; 2.92 s (4CH₃); 3.20 m (CH₂); 3.49 s (OCH₃); 6.90 br.s (NH); ³J_{H,H}=7.1 Hz.

Analogously thereto,

guanidinium chloride is reacted with dimethyl sulfate to give
guanidinium methylsulfate;
   guanidinium chloride is reacted with diethyl sulfate to give
guanidinium ethylsulfate or
   N,N,N′,N′-tetramethyl-N″,N″-diethylguanidinium bromide is reacted with dimethyl sulfate to give
N,N,N′,N′-tetramethyl-N″,N″-diethylguanidinium methylsulfate.

### EXAMPLE 11

### Synthesis of 1-ethyl-3-methylimidazolium methylsulfate

### a) Synthesis of 1-ethyl-3-methylimidazolium bromide

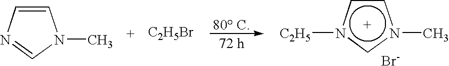

111.43 g (1.36 mol) of methylimidazole and 160 g (1.47 mol) of bromoethane are mixed, and 400 ml of isopropanol are subsequently added. The reaction mixture is heated with stirring for 72 hours, during which the oil-bath temperature is 80° C. Isopropanol is then removed by distillation, and the residue is dried for two hours in vacuo at 13.3 Pa and an oil-bath temperature of 100° C., giving 258.6 g of 1-ethyl-3-methylimidazolium bromide, corresponding to a yield of 99.7%.

M.p.: 73-74° C.

¹H NMR (reference: TMS; CD₃CN), ppm: 1.44 t (CH₃); 3.88 s (CH₃); 4.23 q (CH₂); 7.49 m (CH); 7.56 m (CH); 9.45 br. s. (CH); ³J_{H,H}=7.2 Hz.

### b) Synthesis of 1-ethyl-3-methylimidazolium methylsulfate

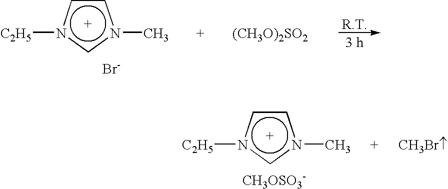

134.17 g (1.064 mol) of dimethyl sulfate are added to 203.26 g (1.064 mol) of 1-ethyl-3-methylimidazolium bromide. The reaction mixture is stirred at room temperature for three hours until the bromide has completely dissolved. The liquid product is subsequently dried for three hours in vacuo at 13.3 Pa and room temperature, giving 236.4 g of 1-ethyl-3-methylimidazolium methylsulfate, corresponding to an approximately quantitative yield.

¹H NMR (reference: TMS; CD₃CN), ppm: 1.44 t (CH₃); 3.50 s (OCH₃); 3.84 (CH₃); 4.18 q (CH₂); 7.40 m (CH); 7.45 m (CH); 8.84 br. s. (CH); ³J_{H,H}=7.3 Hz.

### EXAMPLE 12

### Synthesis of N,N,N′,N′-tetramethyl-S-methylthiouronium ethylsulfate

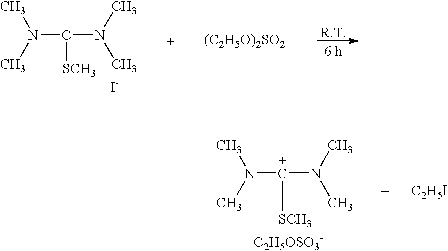

A mixture of 2.25 g (8.21 mmol) of N,N,N′,N′-tetramethyl-S-methylthiouronium iodide and 1.27 g (8.24 mmol) of diethyl sulfate is stirred at room temperature for 6 hours. An NMR measurement indicates the completeness of the reaction. The residue is dried for 1 hour at room temperature in a vacuum of 13.3 Pa, giving 2.16 g of N,N,N′,N′-tetramethyl-S-methylthiouronium ethylsulfate, corresponding to a yield of 96.6%.

¹H NMR (reference: TMS; CD₃CN), ppm: 1.50 t (CH₃); 2.49 s (SCH₃); 3.21 (4CH₃); 3.82 q (CH₂); ³J_{H,H}=7.1 Hz.

Analogously thereto,

N,N,N′,N′-tetramethyl-S-ethylthiouronium iodide is reacted with diethyl sulfate to give
N,N,N′,N′-tetramethyl-S-ethylthiouronium ethylsulfate;
   N,N,N′,N′-tetramethyl-S-propylthiouronium iodide is reacted with dimethyl sulfate to give
N,N,N′,N′-tetramethyl-S-propylthiouronium methylsulfate;
   N,N,N′,N′-tetramethyl-S-butylthiouronium iodide is reacted with diethyl sulfate to give
N,N,N′,N′-tetramethyl-S-butylthiouronium ethylsulfate;
   N,N,N′,N′-tetramethyl-S-octylthiouronium iodide is reacted with diethyl sulfate to give
N,N,N′,N′-tetramethyl-S-octylthiouronium ethylsulfate;
   N,N,N′,N′-tetraethyl-S-methylthiouronium iodide is reacted with diethyl sulfate to give
N,N,N′,N′-tetraethyl-S-methylthiouronium ethylsulfate;
   N,N,N′,N′-tetraethyl-S-ethylthiouronium iodide is reacted with diethyl sulfate to give
N,N,N′,N′-tetraethyl-S-ethylthiouronium ethylsulfate;
   N,N,N′,N′-tetraethyl-S-propylthiouronium iodide is reacted with dimethyl sulfate to give
N,N,N′,N′-tetraethyl-S-propylthiouronium methylsulfate;
   N,N,N′,N′-tetraethyl-S-butylthiouronium iodide is reacted with diethyl sulfate to give
N,N,N′,N′-tetraethyl-S-butylthiouronium ethylsulfate;
   N,N,N′,N′-tetraethyl-S-octylthiouronium iodide is reacted with diethyl sulfate to give
N,N,N′,N′-tetraethyl-S-octylthiouronium ethylsulfate;
   N,N-dimethyl-N′,N′-diethyl-S-methylthiouronium iodide is reacted with diethyl sulfate to give
N,N-dimethyl-N′,N′-diethyl-S-methylthiouronium ethylsulfate;
   N,N-dimethyl-N′,N′-diethyl-S-ethylthiouronium iodide is reacted with diethyl sulfate to give
N,N-dimethyl-N′,N′-diethyl-S-ethylthiouronium ethylsulfate;
   N,N-dimethyl-N′,N′-diethyl-S-propylthiouronium iodide is reacted with dimethyl sulfate to give
N,N-dimethyl-N′,N′-diethyl-S-propylthiouronium methylsulfate;
   N,N-dimethyl-N′,N′-diethyl-S-butylthiouranium iodide is reacted with diethyl sulfate to give
N,N-dimethyl-N′,N′-diethyl-S-butylthiouronium ethylsulfate or
   N,N-dimethyl-N′,N′-diethyl-S-octylthiouronium iodide is reacted with diethyl sulfate to give
N,N-dimethyl-N′,N′-diethyl-S-octylthiouronium ethylsulfate.

### EXAMPLE 13

### Synthesis of 1-butyl-3-methylimidazolium methylsulfate

### a) Synthesis of trimethylsilyl methyl sulfate

(CH₃)₃SiOSO₂Cl+CH₃OH→CH₃OSO₂OSi(CH₃)₃+HCl↑

0.56 g (17.48 mmol) of methanol are added to 3.3 g (17.49 mmol) of the trimethylsilyl ester of chlorosulfonic acid over the course of 10 minutes with stirring and temperature control. All volatile products are removed in a vacuum of 13 Pa at room temperature. 1.21 g of trimethylchlorosilane are added, and the reaction mixture is heated at an oil-bath temperature of 70° C. for 30 minutes. The mixture is subjected to fractional distillation in a vacuum of 13 Pa, giving 2.24 g of trimethylsilyl methyl sulfate of boiling point 63-64° C. The yield corresponds to 69.9%.

¹H NMR (reference: TMS; CD₃CN), ppm: 0.41 s [Si(CH₃)₃]; 3.92 s (CH₃).

### b) Synthesis of 1-butyl-3-methylimidazolium methylsulfate

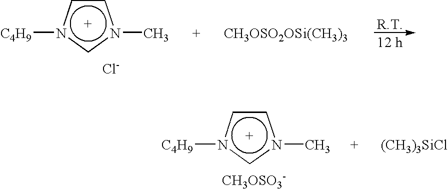

A mixture of 0.91 g (5.21 mmol) of 1-butyl-3-methylimidazolium chloride and 0.96 g (5.21 mmol) of trimethylsilyl methyl sulfate is stirred at room temperature for 12 hours. An NMR measurement shows the completeness of the reaction. The residue is dried for one hour in vacuo at 13.3 Pa and an oil-bath temperature of 60° C., giving 1.30 g of 1-butyl-3-methylimidazolium methylsulfate. The yield is approximately quantitative.

¹H NMR (reference: TMS; CD₃CN), ppm: 0.92 t (CH₃); 1.31 m (CH₂); 1.81 m (CH₂); 3.52 s (OCH₃); 3.86 s (CH₃); 4.16 t (CH₂); 7.43 d, d (CH); 7.47 d, d (CH); 8.90 br. s. (CH); ³J_{H,H}=7.4 Hz; ³J_{H,H}=7.3 Hz; J_{H,H}=1.7 Hz.

### EXAMPLE 14

### Synthesis of 1-butyl-3-methylimidazolium octylsulfate

### a) Synthesis of trimethylsilyl octyl sulfate

(CH₃)₃SiOSO₂Cl+C₈H₁₇OH→C₈H₁₇OSO₂OSi(CH₃)₃+HCl↑

1.17 g (8.98 mmol) of octanol are added to 1.70 g (9.01 mmol) of the trimethylsilyl ester of chlorosulfonic acid. The reaction mixture is stirred at room temperature for 30 minutes, and all volatile products are subsequently removed in a vacuum of 13 Pa and at room temperature. 1.12 g of trimethylchlorosilane are added, and the reaction mixture is heated at an oil-bath temperature of 70° C. for 30 minutes. The mixture is subjected to fractional distillation in a vacuum of 13 Pa, giving 2.48 g of trimethylsilyl octyl sulfate of boiling point 132° C. The yield corresponds to 57.2%.

¹H NMR (reference: TMS; CD₃CN), ppm: 0.40 s [Si(CH₃)₃]; 0.90 m (CH₃); 1.30 m (5CH₂); 1.73 m (CH₂); 4.24 t (CH₂); ³J_{H,H}=6.5 Hz.

### b) Synthesis of 1-butyl-3-methylimidazolium octylsulfate

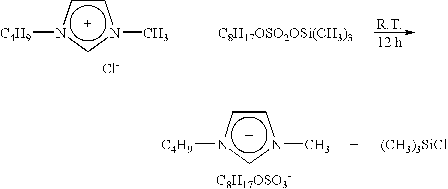

A mixture of 0.358 g (2.05 mmol) of 1-butyl-3-methylimidazolium chloride and 0.58 g (2.06 mmol) of trimethylsilyl octyl sulfate is stirred at room temperature for 12 hours. NMR measurements show the completeness of the reaction. The residue is dried for 1 hour in vacuo at 13.3 Pa and an oil-bath temperature of 60° C., giving 0.71 g of 1-butyl-3-methylimidazolium octylsulfate. The yield is approximately quantitative.

¹H NMR (reference: TMS; CD₃CN), ppm: 0.87 t (CH₃); 0.92 t (CH₃); 1.27 m (5CH₂); 1.31 m (CH₂); 1.54 m (CH₂); 1.81 m (CH₂); 3.81 t (CH₂); 3.87 s (NCH₃); 4.19 t (CH₂); 7.46 d, d (CH); 7.49 d, d (CH); 9.16 br. s. (CH); ³J_{H,H}=7.4 Hz; ³J_{H,H}7.3 Hz; ³J_{H,H}=6.7 Hz; J_{H,H}=1.7 Hz.

1. Process for the preparation of onium alkylsulfates by reaction of an onium halide with a symmetrically substituted dialkyl sulfate, in which the alkyl group can have 1 to 14 C atoms, with an asymmetrically substituted dialkyl sulfate, in which one alkyl group can have 4 to 20 C atoms and the second alkyl group denotes methyl or ethyl, with an alkyl trialkylsilyl sulfate, with an alkyl acyl sulfate or with an alkyl sulfonyl sulfate, where the reaction with a dialkyl sulfate is carried out at room temperature.

2. Process according to claim 1, characterised in that the reaction with a symmetrically substituted dialkyl sulfate, in which the alkyl group can have 1 to 14 C atoms, is carried out at room temperature.

3. Process according to claim 1, characterised in that the reaction with an asymmetrically substituted dialkyl sulfate, in which one alkyl group can have 4 to 20 C atoms and the second alkyl group denotes methyl or ethyl, is carried out at room temperature.

4. Process according to claim 1, characterised in that the reaction is carried out with an alkyl trialkylsilyl sulfate.

5. Process according to claim 1, characterised in that the reaction is carried out with an alkyl acyl sulfate.

6. Process according to claim 1, characterised in that the reaction is carried out with an alkyl sulfonyl sulfate.

7. Process according to claim 1, characterised in that the halide is a phosphonium chloride or bromide, a guanidinium chloride or bromide, a thiouronium chloride, bromide or iodide or a chloride or bromide with a heterocyclic cation.

8. Process according to claim 1, characterised in that the halide conforms to the formula (1)
[PR₄]⁺Hal⁻ (1),
where
Hal denotes Cl or Br and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
where one or more R may be partially or fully substituted by halogens, in particular —F and/or —Cl, or partially by —NO₂, but where all four or three R must not be fully substituted by halogens.

9. Process according to claim 1, characterised in that the halide conforms to the formula (2)
[C(NR¹R²)(NR³R⁴)(NR⁵R⁶)]⁺Hal⁻ (2),
where
Hal denotes Cl or Br and
R¹ to R⁶ each, independently of one another, denotes
hydrogen,
straight-chain or branched alkyl having 1-20 C atoms,
straight-chain or branched alkenyl having 2 to 20 C atoms and one or more double bonds,
where one or more of the substituents R¹ to R⁶ may be partially or fully substituted by halogens, in particular —F and/or —Cl, or partially by —NO₂, but where all substituents on one N atom must not be fully substituted by halogens and
where the substituents R¹ to R⁶ may be connected to one another in pairs by a single or double bond.

10. Process according to claim 1, characterised in that the halide conforms to the formula (3)
[(R¹R²N)—C(═SR⁷)(NR³R⁴)]⁺Hal⁻ (3),
where
Hal denotes Cl, Br or I and
R¹ to R⁷ each, independently of one another, denotes
hydrogen, where hydrogen is excluded for R⁷,
straight-chain or branched alkyl having 1 to 20 C atoms,
straight-chain or branched alkenyl having 2-20 C atoms and one or more double bonds,
where one or more of the substituents R¹ to R⁷ may be partially or fully substituted by halogens, in particular —F and/or —Cl, or partially by —NO₂, but where all substituents on one N atom must not be fully substituted by halogens and
where the substituents R¹ to R⁷ may be connected to one another in pairs by a single or double bond.

11. Process according to claim 1, characterised in that the halide conforms to the formula (4)
[HetN]⁺Hal⁻ (4),
where
Hal denotes Cl or Br and
HetN⁺ denotes a heterocyclic cation selected from the group 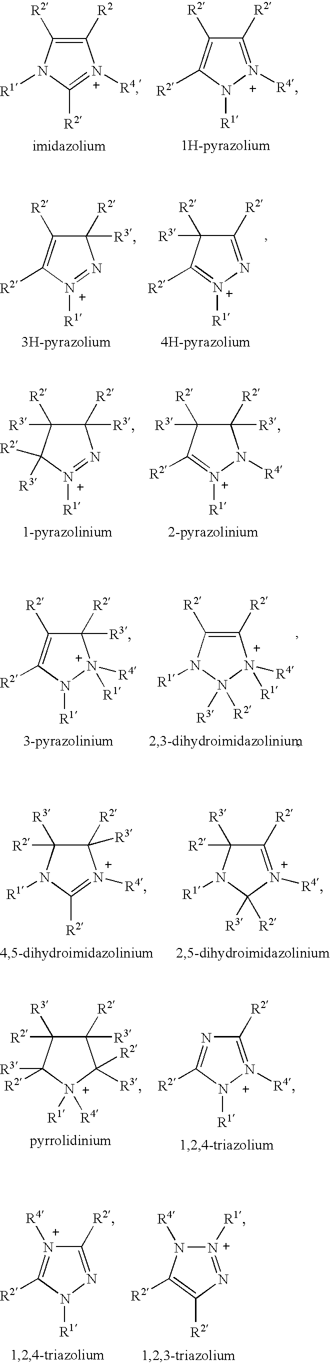 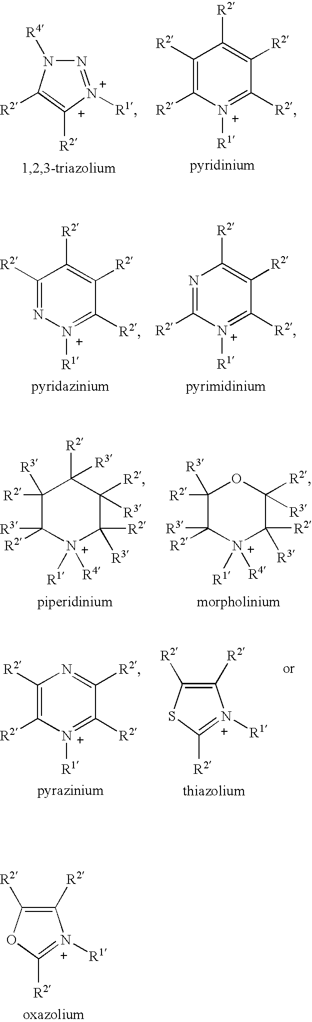
where the substituents
R^{1′} to R^{4′} each, independently of one another, denote
hydrogen or CN,
straight-chain or branched alkyl having 1-20 C atoms, straight-chain or branched alkenyl having 2-20 C atoms and one or more double bonds or
aryl-C₁-C₆-alkyl,
where one or more substituents R^{1′} to R^{4′} may be partially or fully substituted by halogens, in particular —F and/or —Cl, or partially by —NO₂ or CN, but where R^{1′} and R^{4′} must not simultaneously be fully substituted by halogens.

12. Process according to claim 1, characterised in that the reaction of the halide with the dialkyl sulfate, alkyl trialkylsilyl sulfate, alkyl acyl sulfate or alkyl sulfonyl sulfate is carried out without a solvent.

13. One-pot process for the preparation of onium alkylsulfates, in which the alkyl group has 4 to 20 C atoms, characterised in that an onium halide is reacted with a symmetrically substituted dialkyl sulfate, in which the alkyl group can have 1 to 3 C atoms, and an alcohol having 4 to 20 C atoms.

14. Use of the processes according to claim 1 for the purification of onium alkylsulfates which are contaminated by onium halides.

15. Trialkylsilyl octyl sulfates, in which the alkyl group of the trialkylsilyl group can in each case, independently of one another, have 1 to 4 C atoms.

16. Compounds according to claim 15, characterised in that the alkyl groups of the trialkylsilyl group are identical.
